(19) United States
(12) Patent Application Publication
(10) Pub. No.: US 2025/0387095 A1
(43) Pub. Date: Dec. 25, 2025

(54) RADIOGRAPHY SYSTEM, RADIOGRAPHY METHOD, AND STORAGE MEDIUM

(71) Applicant: CANON KABUSHIKI KAISHA, Tokyo (JP)

(72) Inventor: SHIGEO KOBAYASHI, Kanagawa (JP)

(21) Appl. No.: 19/305,619

(22) Filed: Aug. 20, 2025

Related U.S. Application Data
(63) Continuation of application No. PCT/JP2024/005423, filed on Feb. 16, 2024.

(30) Foreign Application Priority Data
Mar. 1, 2023 (JP) 2023-031344

(51) Int. Cl. A61B 6/58; A61B 6/46

(52) U.S. Cl. CPC A61B 6/585; A61B 6/465

(57) ABSTRACT

A radiography system includes a setting unit that sets up an imaging protocol including the orientation of one of a radiation generation device that generates the radiation and a radiation detection device that detects the radiation in the form of an image signal, a detection unit that detects the orientation of the one device, and a determination unit that determines whether the radiography can be performed based on the orientation of the one device included in the imaging protocol set up by the setting unit and the orientation of the one device detected by the detection unit.

### CROSS-REFERENCE TO RELATED APPLICATIONS

This application is a Continuation of International Patent Application No. PCT/JP2024/005423, filed Feb. 16, 2024, which claims the benefit of Japanese Patent Application No. 2023-031344, filed Mar. 1, 2023, both of which are hereby incorporated by reference herein in their entirety.

### BACKGROUND

### Field of the Technology

The present disclosure relates to a radiography system, a radiography method, and a storage medium.

### Description of the Related Art

Nowadays, flat panel detectors (FPDs) made of semiconductor materials are widely used as radiation detectors (hereinafter referred to as “radiation detection devices”) for medical imaging diagnosis or nondestructive inspection using radiation, such as X-rays. In addition, radiography systems are in use that combine such a radiation detection device with a radiation generation device that generate radiation and other devices.

In the radiography systems, the radiation generation device and the radiation detection device (FPD) need to be positioned so as to face each other when radiography is performed. In this case, the radiation generation device and the radiation detection device can be positioned by detecting the orientation of each of the radiation generation device and the radiation detection device. For example, to detect the orientation of the radiation generation device and the radiation detection device, an acceleration sensor or a gyro sensor can be installed in each of the radiation generation device and the radiation detection device, and the orientation can be detected from the acceleration, which is the output value of the acceleration sensor, or the angular velocity, which is the output value of the gyro sensor. Japanese Patent Publication No. 6305085 describes a technique for determining whether a radiation generation device and a radiation detection device (FPD) are facing each other by detecting the orientation of each of the radiation generation device and the radiation detection device (FPD) using the sensor installed in each of the devices.

However, according to the technology described in Japanese Patent Publication No. 6305085, since the sensor for detecting the orientation is provided in each of the radiation generation device and the radiation detection device (FPD), the size and complexity of the overall configuration of the radiography system tend to increase. That is, according to the technology described in Japanese Patent Publication No. 6305085, it is difficult for a radiography system with a simple configuration to determine whether radiography can be performed.

### SUMMARY

Accordingly, the present disclosure provides a system that enables determination as to whether radiography can be performed with a simple configuration.

According to an aspect of the present disclosure, a radiography system for performing radiography using radiation includes a setting unit that sets up an imaging protocol including the orientation of one of a radiation generation device that generates the radiation and a radiation detection device that detects the radiation in a form of an image signal, a detection unit that detects the orientation of the one device, and a determination unit that determines whether the radiography can be performed based on the orientation of the one device included in the imaging protocol set up by the setting unit and the orientation of the one device detected by the detection unit.

The present disclosure also includes a radiography method for use by the radiography system described above and a storage medium including one or more programs for causing a computer to function as each of units of the radiography system described above.

Features of the present disclosure will become apparent from the following description of embodiments with reference to the attached drawings.

### BRIEF DESCRIPTION OF THE DRAWINGS

FIG. 1 illustrates an example of the overall configuration of a radiography system according to an embodiment of the present disclosure.

FIG. 2 illustrates an example of imaging protocol information stored in a storage unit illustrated in FIG. 1, according to a first embodiment of the present disclosure.

FIG. 3 is a sequence diagram illustrating an example of a processing procedure for a radiography method using a radiography system according to the first embodiment of the present disclosure.

FIG. 4 illustrates an example of the radiography performed by the radiography system using an imaging protocol having imaging protocol number information being “1” illustrated in FIG. 2, according to the first embodiment of the present disclosure.

FIG. 5 illustrates an example of the radiography performed by the radiography system using the imaging protocol having the imaging protocol number information being “2” illustrated in FIG. 2, according to the first embodiment of the present disclosure.

FIG. 6 illustrates an example of the radiography performed by the radiography system using the imaging protocol having the imaging protocol number information being “3” illustrated in FIG. 2, according to the first embodiment of the present disclosure.

FIG. 7 illustrates an example of the imaging protocol information stored in the storage unit illustrated in FIG. 1, according to a second embodiment of the present disclosure.

FIG. 8 is a sequence diagram illustrating an example of a processing procedure for a radiography method using a radiography system according to the second embodiment of the present disclosure.

FIG. 9 illustrates an example of the radiography performed by the radiography system using an imaging protocol having imaging protocol number information being “21” illustrated in FIG. 7, according to the second embodiment of the present disclosure.

FIG. 10 illustrates an example of the radiography performed by the radiography system using the imaging protocol having the imaging protocol number information being “22” illustrated in FIG. 7, according to the second embodiment of the present disclosure.

### DESCRIPTION OF THE EMBODIMENTS

Embodiments of the present disclosure are described below with reference to the accompanying drawings. All of the features and the combinations thereof described in the embodiments of the present disclosure below are not necessarily deemed to be essential to every embodiment of the present disclosure.

### First Embodiment

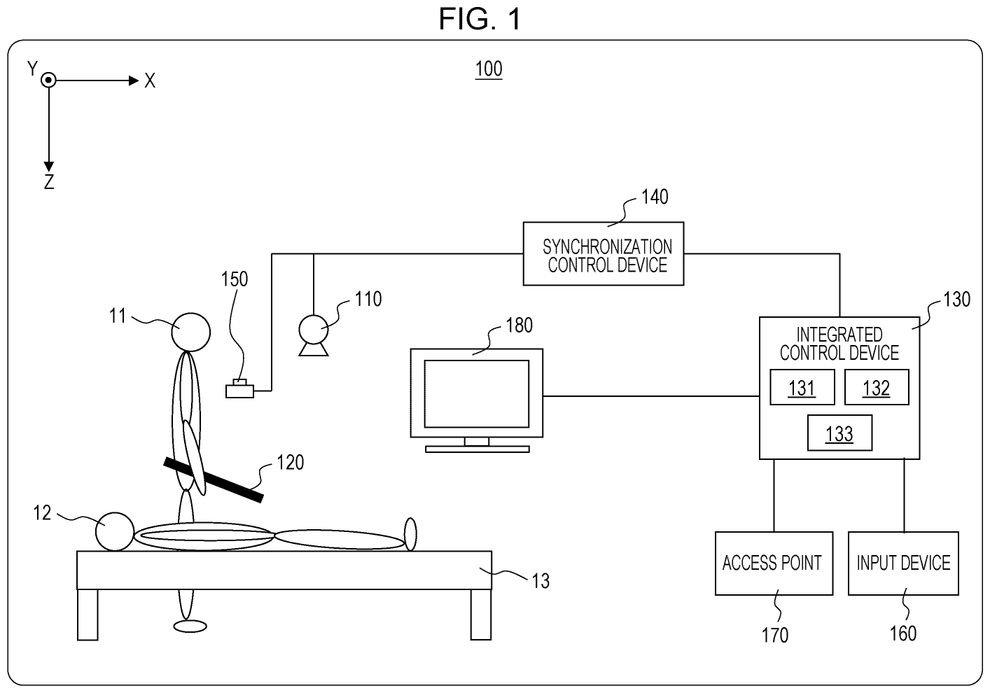

The first embodiment of the present disclosure will now be described with reference to FIGS. 1 to 6.

FIG. 1 illustrates an example of the overall configuration of a radiography system 100 according to the embodiment of the present disclosure. The radiography system 100 is a system for performing radiography of a subject 12 using radiation. As illustrated in FIG. 1, the radiography system 100 includes a radiation generation device 110, a radiation detection device 120, an integrated control device 130, a synchronization control device 140, an exposure switch 150, an input device 160, an access point 170, and a display device 180. FIG. 1 also illustrates the XYZ coordinate system, where the vertical direction is the Z direction, the horizontal direction orthogonal to the Z direction is the X direction, and the direction orthogonal to the Z and X directions is the Y direction.

Also illustrated in FIG. 1 are a radiographer 11, a subject 12, and a radiographic table 13. The radiographer 11 is an examiner who performs radiography of the subject 12. The subject 12 is an examinee undergoing radiography. The radiographic table 13 is a table that supports the subject 12 undergoing radiography. In the example illustrated in FIG. 1, the radiographic table 13 is a table that supports the subject 12 in a lying position.

The radiation generation device 110 is a device that generates radiation directed toward the subject 12 under the control of the integrated control device 130 and the synchronization control device 140. To generate radiation, such as X-rays, the radiation generation device 110 includes a radiation tube that accelerates electrons by high voltage and bombards the electrons with an anode, for example. According to the present embodiment, the radiation is not limited to X-rays, but may be α-rays, β-rays, γ-rays, neutron rays, or the like.

When performing radiography of the subject 12, the radiation detection device 120 is disposed by the radiographer 11 so as to face the radiation generation device 110 with the subject 12 therebetween. When performing radiography of the subject 12, the radiation detection device 120 detects the incident radiation (including radiation transmitted through the subject 12) in the form of an image signal, which is an electrical signal, and generates a radiation image on the basis of the detected image signal. According to the present embodiment, the radiation detection device 120 can be configured as, for example, a radiation detector composed of the FPD described above.

The integrated control device 130 is a device that performs overall control of the operation of the radiography system 100 and performs a variety of types of processing. The integrated control device 130 controls each of the devices of the radiography system 100 and performs a variety of types of processing on the basis of information input from, for example, the input device 160 and the access point 170. For example, the integrated control device 130 sets up an imaging protocol for radiography on the basis of the information input from the input device 160 and controls displaying of a radiation image generated by the radiation detection device 120 on the display device 180. In addition, for example, the integrated control device 130 communicates setting information to enable wireless communication with the radiation detection device 120 via the access point 170. The integrated control device 130 further includes a setting unit 131, a determination unit 132, and a storage unit 133, as illustrated in FIG. 1. The internal constituent units 131 to 133 of the integrated control device 130 are described below.

The synchronization control device 140 is a device that includes a circuit that mediates communication and monitors the status of each of the radiation detection device 120 and the radiation generation device 110 under the control of the integrated control device 130. For example, the synchronization control device 140 controls emission of radiation from the radiation generation device 110 and radiography of the subject 12 by the radiation detection device 120 under the control of the integrated control device 130. The synchronization control device 140 may also include a built-in HUB or the like that connects a plurality of network devices.

The exposure switch 150 is a switch operated by the radiographer 11 to irradiate the subject 12 with radiation from the radiation generation device 110 when the radiographer 11 needs to perform radiography of the subject 12.

The input device 160 is a device for a user, such as the radiographer 11, to input a variety of types of information to the integrated control device 130.

The access point 170 is a radio relay device for wireless communication between the integrated control device 130 and the radiation detection device 120.

The display device 180 is a device that displays a variety of information and a variety of images under the control of the integrated control device 130.

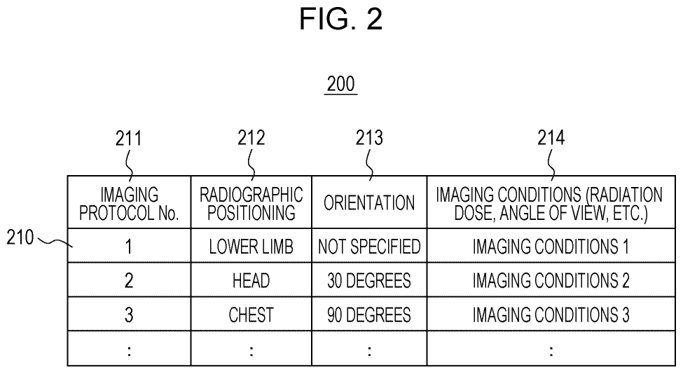

FIG. 2 illustrates an example of imaging protocol information 200 stored in the storage unit 133 illustrated in FIG. 1, according to the first embodiment of the present disclosure.

The imaging protocol information 200 illustrated in FIG. 2 includes a plurality of imaging protocols 210. Each of the imaging protocols 210 has, set therein, imaging protocol number information 211, radiographic positioning information 212, orientation information 213, and imaging condition information 214 that includes, for example, the radiation dose and the imaging angle of view. According to the first embodiment, the orientation information 213 is information indicating the orientation of the radiation detection device 120, which is paired with the radiation generation device 110. Furthermore, the orientation information 213 of the radiation detection device 120 illustrated in FIG. 2 is information indicating the angle of inclination of the radiation detection device 120 with respect to the X direction that indicates the horizontal direction in FIG. 1 and that indicates an angle of inclination of 0 degrees (the angle measured in a clockwise direction from the X direction (to the Z direction) is a positive (+) angle).

The imaging protocols 210 illustrated in FIG. 2 are described in more detail below.

In the imaging protocol 210 having the imaging protocol number information 211 being “1”, “lower limb” is set as the radiographic positioning information 212, “not specified” is set as the orientation information 213 of the radiation detection device 120, and “imaging conditions 1” is set as the imaging condition information 214. In the imaging protocol 210 having the imaging protocol number information 211 being “2”, “head” is set as the radiographic positioning information 212, “30 degrees” is set as the orientation information 213 of the radiation detection device 120, and “imaging conditions 2” is set as the imaging condition information 214. In the imaging protocol 210 having the imaging protocol number information 211 being “3”, “chest” is set as the radiographic positioning information 212, “90 degrees” is set as the orientation information 213 of the radiation detection device 120, and “imaging conditions 3” is set as the imaging condition information 214.

The setting unit 131 of the integrated control device 130 illustrated in FIG. 1 is a setting unit that stores the imaging protocol information 200 including the plurality of imaging protocols 210 illustrated in FIG. 2 in the storage unit 133 to set up the imaging protocol information 200.

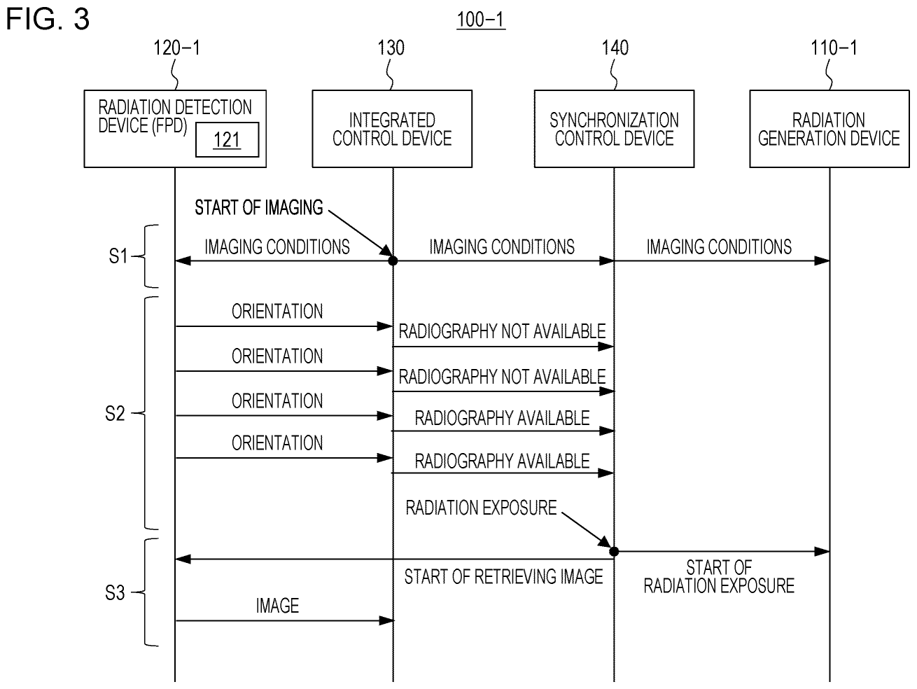

FIG. 3 is a sequence diagram illustrating an example of the processing procedure for a radiography method for use by the radiography system 100 according to the first embodiment of the present disclosure. In the following description, as illustrated in FIG. 3, the radiography system 100 according to the first embodiment is referred to as a “radiography system 100-1”. In FIG. 3, the configurations similar to those illustrated in FIG. 1 are identified by the same reference numerals, and detailed descriptions of the configurations are omitted.

Illustrated in FIG. 3 are the process flows of the radiation generation device 110, the radiation detection device 120, the integrated control device 130, and the synchronization control device 140 among the devices constituting the radiography system 100-1 according to the first embodiment. In the following description, as illustrated in FIG. 3, the radiation generation device 110 according to the first embodiment is referred to as a “radiation generation device 110-1”, and the radiation detection device 120 according to the first embodiment is referred to as a “radiation detection device 120-1”

As illustrated in FIG. 3, the radiation detection device 120-1 according to the first embodiment includes an orientation detection sensor 121 for detecting the orientation of the radiation detection device 120-1. More specifically, the orientation detection sensor 121 is a detection unit that detects, as the orientation of the radiation detection device 120-1, the angle of inclination of the radiation detection device 120-1 with respect to the X direction that indicates the horizontal direction in FIG. 1 and that indicates an angle of inclination of 0 degrees, like the orientation information 213 illustrated in FIG. 2.

In FIG. 3, step S1 indicates the process of starting imaging, step S2 indicates the process of detecting the orientation, and step S3 indicates the process of radiography. At this time, it is assumed that prior to starting the processing illustrated in FIG. 3, the imaging protocol information 200, which includes the plurality of imaging protocols 210 illustrated in FIG. 2, is stored in the storage unit 133 and is set up by the setting unit 131 of the integrated control device 130 illustrated in FIG. 1. In addition to storing the imaging protocol information 200 described above, the storage unit 133 of the integrated control device 130 illustrated in FIG. 1 stores various information and programs necessary for the integrated control device 130 to perform the processing illustrated in FIG. 3.

In step S1, the integrated control device 130 first selects the imaging protocol 210 to be used for radiography from among the plurality of imaging protocols 210 stored in the storage unit 133 on the basis of, for example, information input from the input device 160 and starts radiography. Subsequently, the integrated control device 130 transmits the imaging condition information 214 set in the selected imaging protocol 210 to the radiation detection device 120-1 and the synchronization control device 140. Furthermore, upon receiving the imaging condition information 214 from the integrated control device 130, the synchronization control device 140 notifies the radiation generation device 110-1 of the required imaging condition information.

In step S2, the integrated control device 130 periodically acquires the information indicating the orientation of the radiation detection device 120-1 from the orientation detection sensor 121 of the radiation detection device 120-1. According to the first embodiment, the determination unit 132 of the integrated control device 130 is a determination unit for determining whether radiography of the subject 12 can be performed using the information indicating the orientation of the radiation detection device 120-1 acquired from the orientation detection sensor 121. More specifically, the determination unit 132 compares the orientation indicated by the orientation information 213 in the imaging protocol 210 selected in step S1 with the orientation of the radiation detection device 120-1 acquired from the orientation detection sensor 121 and determines whether radiography can be performed on the basis of the result of the comparison. Still more specifically, the determination unit 132 determines that radiography can be performed if the difference between the orientation indicated by the orientation information 213 in the imaging protocol 210 selected in step S1 and the orientation of the radiation detection device 120-1 acquired from the orientation detection sensor 121 is within a predetermined range. However, the determination unit 132 determines that radiography cannot be performed if the difference between the orientation indicated by the orientation information 213 in the imaging protocol 210 selected in step S1 and the orientation of the radiation detection device 120-1 acquired from the orientation detection sensor 121 is not within the predetermined range.

If the determination unit 132 determines that radiography of the subject 12 cannot be performed, the integrated control device 130 notifies the synchronization control device 140 that radiography cannot be performed. When the determination unit 132 determines that radiography of the subject 12 cannot be performed, the integrated control device 130 causes the display device 180 to display “Radiography not available” as information indicating the result of determination as to whether radiography can be performed. Furthermore, the integrated control device 130 causes the display device 180 to display information indicating the difference between the orientation of the radiation detection device 120-1 in the imaging protocol 210 and the orientation detected by the orientation detection sensor 121.

However, if the determination unit 132 determines that radiography of the subject 12 can be performed, the integrated control device 130 notifies the synchronization control device 140 that radiography can be performed. In addition, when the determination unit 132 determines that radiography of the subject 12 can be performed, the integrated control device 130 causes the display device 180 to display “Radiography available” as information indicating the result of determination as to whether radiography can be performed. Still furthermore, the integrated control device 130 causes the display device 180 to display information indicating the difference between the orientation of the radiation detection device 120-1 in the imaging protocol 210 and the orientation detected by the orientation detection sensor 121.

As illustrated in FIG. 3, in step S2, the determination unit 132 determines whether radiography of the subject 12 can be performed each time the information indicating the orientation of the radiation detection device 120-1 is acquired from the orientation detection sensor 121 of the radiation detection device 120-1.

In step S3, when the exposure switch 150 is pressed by the radiographer 11, the synchronization control device 140 detects this event. The synchronization control device 140 then controls emission of radiation from the radiation generation device 110-1 according to the result of determination as to whether radiography can be performed at the time the exposure switch 150 is pressed. More specifically, if the result of determination as to whether radiography can be performed at the time the exposure switch 150 is pressed is “Radiography not available,” the synchronization control device 140 performs control to continue the stoppage of emission of radiation from the radiation generation device 110-1 without sending any notification to the radiation generation device 110-1. However, if the result of determination as to whether radiography can be performed at the time the exposure switch 150 is pressed is “Radiography available,” the synchronization control device 140 notifies the radiation generation device 110-1 of the start of radiation exposure and performs control to start the emission of radiation from the radiation generation device 110-1. In addition, if the result of determination as to whether radiography can be performed at the time the exposure switch 150 is pressed is “Radiography available”, the synchronization control device 140 notifies the radiation detection device 120-1 of the start of retrieving the radiation image via the integrated control device 130. Thereafter, upon completion of retrieving the radiation image, the radiation detection device 120-1 transmits the retrieved radiation image to the integrated control device 130.

When the processing in step S3 is completed, the process of the sequence diagram illustrated in FIG. 3 ends.

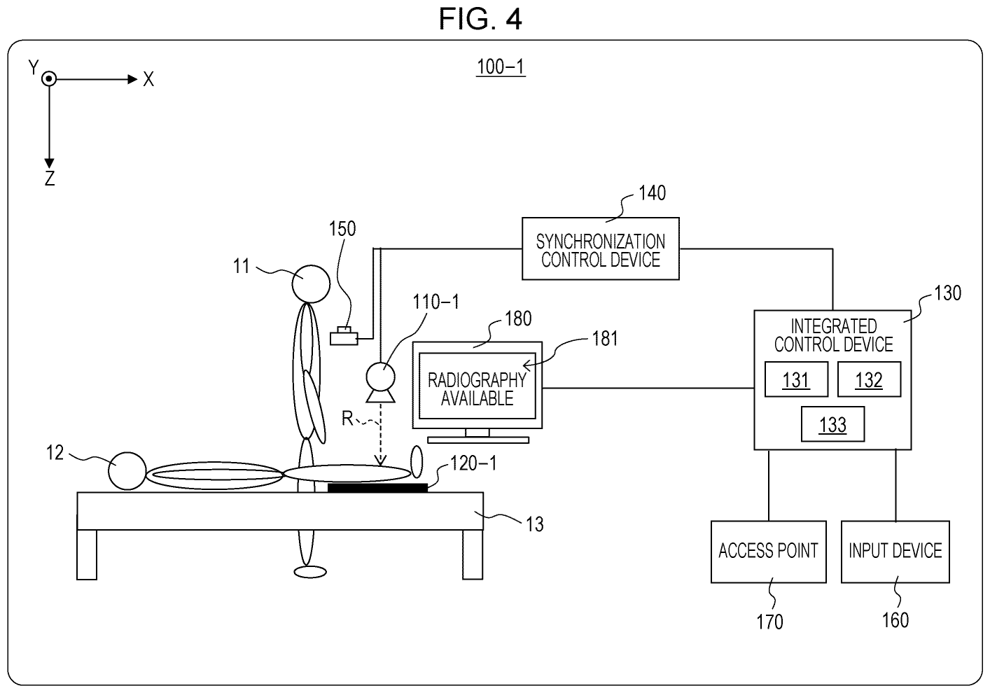

The processing performed for each of the plurality of imaging protocols 210 illustrated in FIG. 2 is described below with reference to FIGS. 4 to 6.

FIG. 4 illustrates an example of the radiography performed by the radiography system 100-1 using the imaging protocol 210 having the imaging protocol number information 211 being “1” illustrated in FIG. 2, according to the first embodiment of the present disclosure. In FIG. 4, the configurations similar to those illustrated in FIGS. 1 and 3 are identified by the same reference numerals, and detailed descriptions of the configurations are omitted. FIG. 4 also illustrates the XYZ coordinate system corresponding to the XYZ coordinate system illustrated in FIG. 1. In FIG. 4, as illustrated in FIG. 3, the radiation detection device 120-1 includes the orientation detection sensor 121 for detecting the orientation of the radiation detection device 120-1. Furthermore, in FIG. 4, the radiographer 11 installs the radiation detection device 120-1 and adjusts the orientation of the radiation generation device 110-1.

In the imaging protocol 210 having the imaging protocol number information 211 being “1” illustrated in FIG. 2, “lower limb” is set as the radiographic positioning information 212, “not specified” is set as the orientation information 213 of the radiation detection device 120-1, and “imaging conditions 1” is set as the imaging condition information 214. The imaging protocol 210 having the imaging protocol number information 211 being “1” illustrated in FIG. 2 is used when the radiation detection device 120-1 is placed on the radiographic table 13 during radiography of the lower limb of the subject 12. In the imaging protocol 210 having the imaging protocol number information 211 being “1” illustrated in FIG. 2, the orientation of the radiation detection device 120-1 is set to “not specified,” which means that the orientation of the radiation detection device 120-1 is not restricted.

Assume that in step S1 illustrated in FIG. 3, the imaging protocol 210 having the imaging protocol number information 211 being “1” illustrated in FIG. 2 is selected, and radiography is started. In this case, in step S2 illustrated in FIG. 3, the integrated control device 130 notifies the synchronization control device 140 that radiography can be performed. In addition, as illustrated in FIG. 4, the integrated control device 130 causes the display device 180 to display “Radiography available” as information 181 on the result of determination as to whether radiography can be performed. This enables the radiographer 11 to perform radiography using the exposure switch 150 after checking the information 181 (“Radiography available”) that is displayed on the display device 180 to indicate the determination result of the availability of radiography.

Subsequently, in step S3 illustrated in FIG. 3, if the exposure switch 150 is pressed by the radiographer 11, the synchronization control device 140 notifies the radiation generation device 110-1 of the start of radiation exposure and performs control to start emission of radiation R from the radiation generation device 110-1. Furthermore, since the result of determination as to whether radiography can be performed at the time the exposure switch 150 is pressed is “Radiography available”, the synchronization control device 140 notifies the radiation detection device 120-1 of start of retrieving the radiation image via the integrated control device 130. Thereafter, upon completion of retrieving the radiation image, the radiation detection device 120-1 transmits the retrieved radiation image to the integrated control device 130.

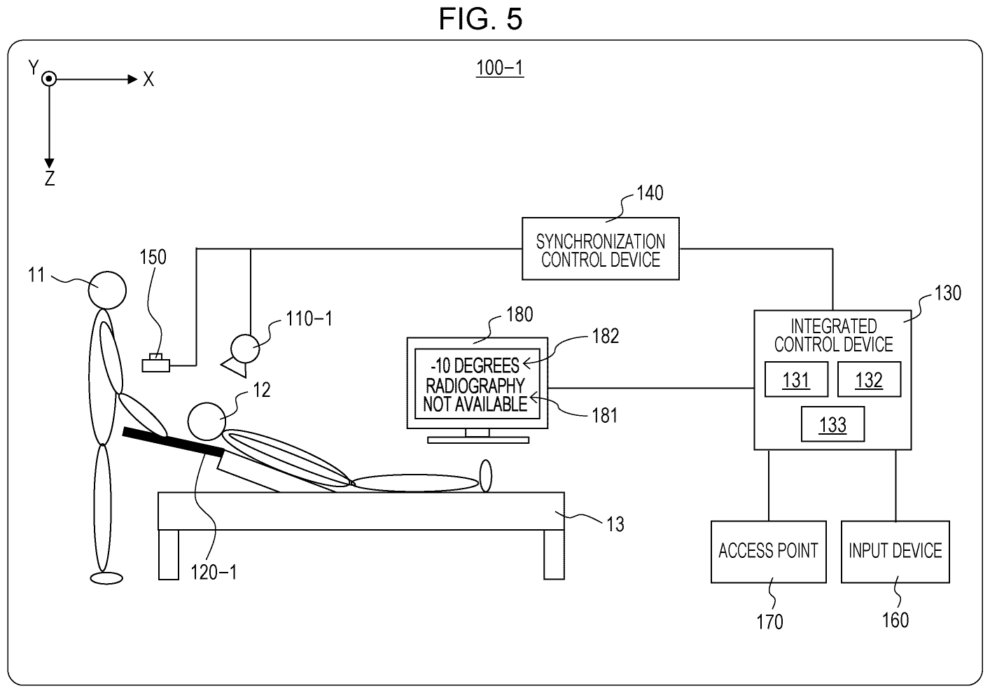

FIG. 5 illustrates an example of the radiography performed by the radiography system 100-1 using the imaging protocol 210 having the imaging protocol number information 211 being “2” illustrated in FIG. 2, according to the first embodiment of the present disclosure. In FIG. 5, the configurations similar to those illustrated in FIGS. 1, 3, and 4 are identified by the same reference numerals, and detailed descriptions of the configurations are omitted. FIG. 5 also illustrates the XYZ coordinate system corresponding to the XYZ coordinate system illustrated in FIGS. 1 and 4. In FIG. 5, as illustrated in FIG. 3, the radiation detection device 120-1 includes the orientation detection sensor 121 for detecting the orientation of the radiation detection device 120-1. Furthermore, in FIG. 5, the radiographer 11 installs the radiation detection device 120-1 and adjusts the orientation of the radiation generation device 110-1.

In the imaging protocol 210 having imaging protocol number information 211 being “2” illustrated in FIG. 2, “head” is set as the radiographic positioning information 212, “30 degrees” is set as the orientation information 213 of the radiation detection device 120-1, and “imaging conditions 2” is set as the imaging condition information 214. The imaging protocol 210 having the imaging protocol number information 211 being “2” illustrated in FIG. 2 is used when the radiation detection device 120-1 is in contact with the head of the subject 12 during radiography of the head of the subject 12. In the imaging protocol 210 having the imaging protocol number information 211 being “2” illustrated in FIG. 2, the orientation of the radiation detection device 120-1 is set to 30 degrees so as to match the inclinations of the radiographic table 13, the subject 12, and the radiation generation device 110-1. In the example illustrated in FIG. 5, the orientation of the radiation detection device 120-1 is information indicating the angle of inclination of the radiation detection device 120-1 with respect to the X direction that indicates the horizontal direction and that indicates an angle of inclination of 0 degrees (the angle measured in a clockwise direction from the X direction (to the Z direction) is a positive (+) angle). In addition, in the example illustrated in FIG. 5, the predetermined range used by the determination unit 132 of the integrated control device 130 to determine whether radiography can be performed is set to ±5 degrees, for example. That is, in this case, the determination unit 132 determines that radiography can be performed when the angle indicating the orientation of the radiation detection device 120-1 acquired from the orientation detection sensor 121 is in the range of 25 degrees to 35 degrees (inclusive) and determines that radiography cannot be performed when the angle is not in the range of 25 degrees to 35 degrees.

In step S1 illustrated in FIG. 3, the imaging protocol 210 having the imaging protocol number information 211 being “2” illustrated in FIG. 2 is selected, and radiography is started. In this case, in step S2 illustrated in FIG. 3, the determination unit 12 can be performed. More specifically, the determination unit 132 determines whether radiography can be performed on the basis of the difference between the orientation information 213 (30 degrees) in the imaging protocol 210 having the imaging protocol number information 211 being “2” and the orientation of the radiation detection device 120-1 acquired from the orientation detection sensor 121. In the example illustrated in FIG. 5, the orientation of the radiation detection device 120-1 acquired from the orientation detection sensor 121 is 20 degrees, and it is determined that radiography cannot be performed on the basis of the difference between 20 degrees and the orientation information 213 (30 degrees) in the imaging protocol 210 having the imaging protocol number information 211 being “2”. That is, the determination unit 132 determines that radiography of the subject 12 cannot be performed because the difference is-10 degrees, which is not in the predetermined range of ±5 degrees.

If the determination unit 132 determines that radiography of the subject 12 cannot be performed, the integrated control device 130 notifies the synchronization control device 140 that radiography cannot be performed. In addition, when the determination unit 132 determines that radiography of the subject 12 cannot be performed, the integrated control device 130 causes the display device 180 to display “Radiography not available” as the information 181 indicating the result of determination as to whether radiography can be performed, as illustrated in FIG. 5. Furthermore, the integrated control device 130 causes the display device 180 to display “−10 degrees” as information 182 indicating the difference between the orientation of the radiation detection device 120-1 in the imaging protocol 210 and the orientation detected by the orientation detection sensor 121, as illustrated in FIG. 5. As a result, the radiographer 11 can check the information 181 indicating the result of determination as to whether radiography can be performed (radiography not available) and the information 182 indicating the difference in the orientations of the radiation detection device 120-1 displayed on the display device 180 and, thereafter, adjust the installation position of the radiation detection device 120-1. That is, according to the first embodiment, the positioning of the radiation detection device 120-1 necessary for radiography can be performed with a simple configuration.

Subsequently, when the radiographer 11 adjusts the installation position of the radiation detection device 120-1 so that the angle indicating the orientation of the radiation detection device 120-1 acquired from the orientation detection sensor 121 is within the range of 25 degrees to 35 degrees, the difference described above is set within the predetermined range (±5 degrees). In this case, the determination unit 132 determines that radiography of the subject 12 can be performed. When the determination unit 132 determines that radiography of the subject 12 can be performed, the integrated control device 130 notifies the synchronization control device 140 that radiography can be performed. In addition, when the determination unit 132 determines that radiography of the subject 12 can be performed, the integrated control device 130 causes the display device 180 to display “Radiography available” as the information 181 indicating the result of determination as to whether radiography can be performed. Furthermore, the integrated control device 130 causes the display device 180 to display the information 182 indicating the difference between the orientation of the radiation detection device 120-1 in the imaging protocol 210 and the orientation detected by the orientation detection sensor 121. This enables the radiographer 11 to perform radiography using the exposure switch 150 after checking the information 181 (“Radiography available”) that is displayed on the display device 180 to indicate the determination result of the availability of radiography.

Then, in step S3 illustrated in FIG. 3, when the exposure switch 150 is pressed by the radiographer 11, the synchronization control device 140 notifies the radiation generation device 110-1 of the start of radiation exposure and performs control to start emission of radiation R from the radiation generation device 110-1. In addition, since the result of determination as to whether radiography can be performed at the time the exposure switch 150 is pressed is “Radiography available,” the synchronization control device 140 notifies the radiation detection device 120-1 of the start of retrieving a radiation image via the integrated control device 130. Thereafter, upon completion of retrieving a radiation image, the radiation detection device 120-1 transmits the retrieved radiation image to the integrated control device 130.

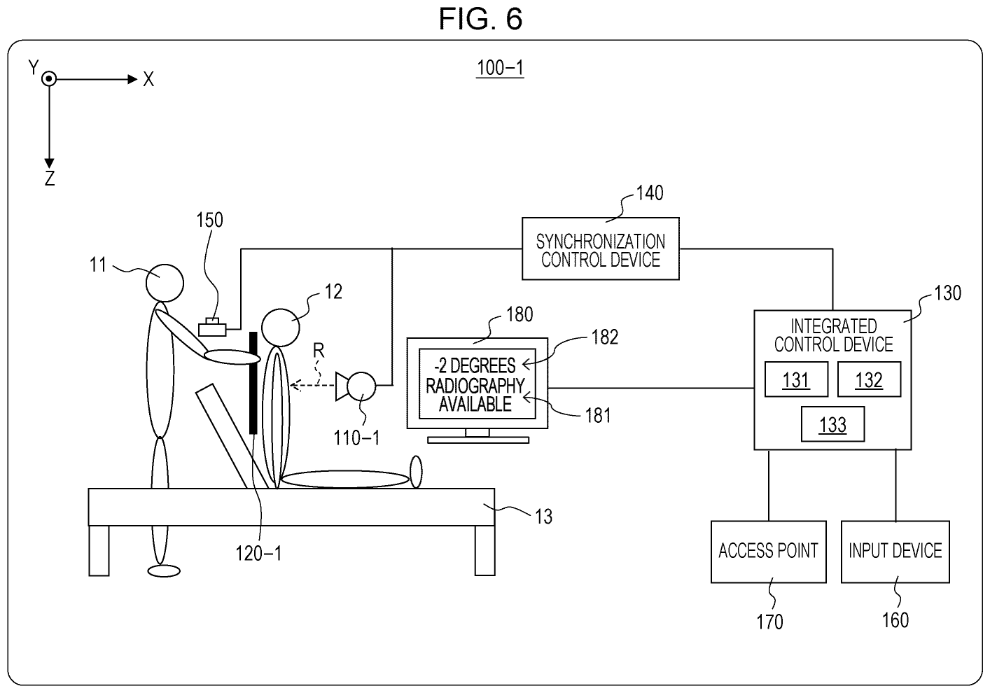

FIG. 6 illustrates an example of the radiography performed by the radiography system 100-1 using the imaging protocol 210 having the imaging protocol number information 211 being “3” illustrated in FIG. 2, according to the first embodiment of the present disclosure. In FIG. 6, the configurations similar to those illustrated in FIG. 1 and FIGS. 3 to 5 are identified by the same reference numerals, and detailed descriptions of the configurations are omitted. FIG. 6 also illustrates the XYZ coordinate system corresponding to the XYZ coordinate system illustrated in FIGS. 1, 4, and 5. In FIG. 6, as illustrated in FIG. 3, the radiation detection device 120-1 includes the orientation detection sensor 121 for detecting the orientation of the radiation detection device 120-1. Furthermore, in FIG. 6, the radiographer 11 installs the radiation detection device 120-1 and adjusts the orientation of the radiation generation device 110-1.

In the imaging protocol 210 having the imaging protocol number information 211 being “3” illustrated in FIG. 2, “chest” is set as the radiographic positioning information 212, “90 degrees” is set as the orientation information 213 of the radiation detection device 120-1, and “imaging conditions 3” is set as the imaging condition information 214. The imaging protocol 210 having the imaging protocol number information 211 being “3” illustrated in FIG. 2 is used when the radiation detection device 120-1 is in contact with the chest region of the subject 12 during radiography of the chest of the subject 12. In the imaging protocol 210 having the imaging protocol number information 211 being “3” illustrated in FIG. 2, the orientation of the radiation detection device 120-1 is set to “90 degrees” (the Z direction) so as to match the inclinations of the radiographic table 13, the subject 12, and the radiation generation device 110-1. In the example illustrated in FIG. 6, the orientation of the radiation detection device 120-1 is information indicating the angle of inclination of the radiation detection device 120-1 with respect to the X direction that indicates the horizontal direction and that indicates an angle of inclination of 0 degrees (the angle measured in a clockwise direction from the X direction (to the Z direction) is a positive (+) angle). In addition, in the example illustrated in FIG. 6, the predetermined range used by the determination unit 132 of the integrated control device 130 to determine whether radiography can be performed is set to ±5 degrees, for example. That is, in this case, the determination unit 132 determines that radiography can be performed if the angle indicating the orientation of the radiation detection device 120-1 acquired from the orientation detection sensor 121 is in the range of 85 degrees to 95 degrees (inclusive) and determines that radiography cannot be performed if the angle is not in the range of 85 degrees to 95 degrees.

In step S1 illustrated in FIG. 3, the imaging protocol 210 having the imaging protocol number information 211 being “3” illustrated in FIG. 2 is selected, and radiography is started. In this case, in step S2 illustrated in FIG. 3, the determination unit 12 can be performed. More specifically, the determination unit 132 determines whether radiography can be performed on the basis of the difference between the orientation information 213 (90 degrees) in the imaging protocol 210 having the imaging protocol number information 211 being “3” and the orientation of the radiation detection device 120-1 acquired from the orientation detection sensor 121. In the example illustrated in FIG. 6, the orientation of the radiation detection device 120-1 acquired from the orientation detection sensor 121 is 88 degrees, and it is determined that radiography can be performed on the basis of the difference between 88 degrees and the orientation information 213 (90 degrees) in the imaging protocol 210 having the imaging protocol number information 211 being “3”. That is, the determination unit 132 determines that radiography of the subject 12 can be performed because the difference is-2 degrees, which is in the predetermined range of ±5 degrees.

If the determination unit 132 determines that radiography of the subject 12 can be performed, the integrated control device 130 notifies the synchronization control device 140 that radiography can be performed. In addition, when the determination unit 132 determines that radiography of the subject 12 can be performed, the integrated control device 130 causes the display device 180 to display “Radiography available” as the information 181 indicating the result of determination as to whether radiography can be performed, as illustrated in FIG. 6. Furthermore, the integrated control device 130 causes the display device 180 to display “−2 degrees” as the information 182 indicating the difference between the orientation of the radiation detection device 120-1 in the imaging protocol 210 and the orientation detected by the orientation detection sensor 121, as illustrated in FIG. 6. This enables the radiographer 11 to perform radiography using the exposure switch 150 after checking the information 181 (“Radiography available”) that is displayed on the display device 180 to indicate the determination result of the availability of radiography.

In step S3 illustrated in FIG. 3, when the exposure switch 150 is pressed by the radiographer 11, the synchronization control device 140 notifies the radiation generation device 110-1 of the start of radiation exposure and performs control to start emission of radiation R from the radiation generation device 110-1. In addition, since the result of determination as to whether radiography can be performed at the time the exposure switch 150 is pressed is “Radiography available,” the synchronization control device 140 notifies the radiation detection device 120-1 of the start of retrieving a radiation image via the integrated control device 130. Thereafter, upon completion of retrieving the radiation image, the radiation detection device 120-1 transmits the retrieved radiation image to the integrated control device 130.

According to the first embodiment, the radiography system 100-1 includes the setting unit 131 that sets up the imaging protocols 210 containing the orientation of the radiation detection device 120-1 which is paired with the radiation generation device 110-1 required for radiography. The radiography system 100-1 further includes the orientation detection sensor 121, which is a detection unit for detecting the orientation of the radiation detection device 120-1. Still furthermore, the radiography system 100-1 includes the determination unit 132 that determines whether radiography can be performed on the basis of the orientation of the radiation detection device included in the imaging protocol 210 set by the setting unit 131 and the orientation of the radiation detection device 120-1 detected by the orientation detection sensor 121.

Such a simple configuration enables determination as to whether radiography can be performed.

### Second Embodiment

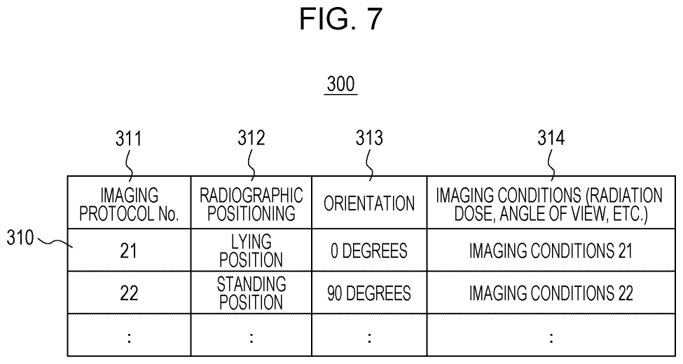

The second embodiment of the present disclosure is described below with reference to FIGS. 7 to 10. In the description of the second embodiment below, description of matters that are the same as those according to the first embodiment above are omitted, and only the matters that differ from those according to the first embodiment described are described.

The overall configuration of a radiography system 100 according to the second embodiment is the same as the overall configuration of the radiography system 100 according to the embodiment of the present disclosure illustrated in FIG. 1.

FIG. 7 illustrates an example of imaging protocol information 300 stored in the storage unit 133 illustrated in FIG. 1 according to the second embodiment of the present disclosure.

The imaging protocol information 300 illustrated in FIG. 7 includes a plurality of imaging protocols 310. Each of the imaging protocols 310 has, as setting information, imaging protocol number information 311, radiographic positioning information 312, orientation information 313, and imaging condition information 314 that includes the radiation dose, the imaging angle of view, and the like. According to the second embodiment, the orientation information 313 is information indicating the orientation of the radiation generation device 110, which is paired with the radiation detection device 120. Furthermore, the orientation information 313 of the radiation generation device 110 illustrated in FIG. 7 is information indicating the angle of inclination of the radiation generation device 110 with respect to the Z direction that indicates the vertical direction in FIG. 1 and that indicates an angle of inclination of 0 degrees (the angle measured in a counterclockwise direction from the Z direction (to the X direction) is a positive (+) angle).

Each of the imaging protocols 310 illustrated in FIG. 7 is described in detail below.

In the imaging protocol 310 having the imaging protocol number information 311 being “21, “lying position” is set as the radiographic positioning information 312, “0 degrees” is set as the orientation information 313 of the radiation generation device 110, and “imaging conditions 21” is set as the imaging condition information 314. In the imaging protocol 310 having the imaging protocol number information 311 being “22,” “standing position” is set as the radiographic positioning information 312, “90 degrees” is set as the orientation information 313 of the radiation generation device 110, and “imaging conditions 22” is set as the imaging condition information 314.

The setting unit 131 of the integrated control device 130 illustrated in FIG. 1 is a setting unit that stores the imaging protocol information 300 including the plurality of imaging protocols 310 illustrated in FIG. 7 in the storage unit 133 and that sets up the imaging protocol information 300.

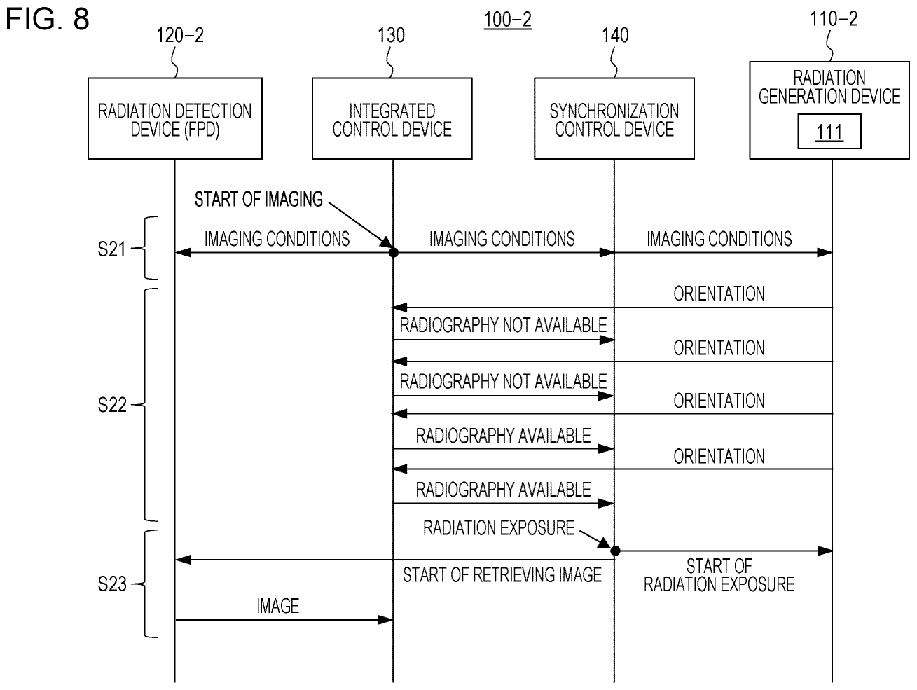

FIG. 8 is a sequence diagram illustrating an example of a processing procedure for a radiography method using the radiography system 100 according to the second embodiment of the present disclosure. In the following description, as illustrated in FIG. 8, the radiography system 100 according to the second embodiment is referred to as a “radiography system 100-2”. In FIG. 8, the configurations similar to those illustrated in FIG. 1 are identified by the same reference numerals, and detailed descriptions of the configurations are omitted.

Illustrated in FIG. 8 are the process flows of the radiation generation device 110, the radiation detection device 120, the integrated control device 130, and the synchronization control device 140 among the devices constituting the radiography system 100-2 according to the second embodiment. In the following description, the radiation generation device 110 according to the second embodiment is referred to as a “radiation generation device 110-2” and the radiation detection device 120 according to the second embodiment is referred to as a “radiation detection device 120-2” as illustrated in FIG. 8.

As illustrated in FIG. 8, the radiation generation device 110-2 according to the second embodiment includes an orientation detection sensor 111 for detecting the orientation of the radiation generation device 110-2. More specifically, the orientation detection sensor 111 is a detection unit that detects, as the orientation of the radiation generation device 110-2, the angle of inclination of the radiation generation device 110-2 with respect to the Z direction that indicates the vertical direction in FIG. 1 and that indicates an angle of inclination of 0 degrees, like the orientation information 313 illustrated in FIG. 7.

In FIG. 8, step S21 indicates the process of starting imaging, step S22 indicates the process of detecting the orientation, and step S23 indicates the process of radiography. At this time, it is assumed that prior to starting the processing illustrated in FIG. 8, the imaging protocol information 300, which includes the plurality of imaging protocols 310 illustrated in FIG. 7, is stored in the storage unit 133 and is set up by the setting unit 131 of the integrated control device 130 illustrated in FIG. 1. In addition to storing the imaging protocol information 300 described above, the storage unit 133 of the integrated control device 130 illustrated in FIG. 1 stores various information and programs necessary for the integrated control device 130 to perform the processing illustrated in FIG. 8.

In step S21, the integrated control device 130 first selects the imaging protocol 310 to be used for radiography from among the plurality of imaging protocols 310 stored in the storage unit 133 on the basis of, for example, information input from the input device 160 and starts radiography. Subsequently, the integrated control device 130 transmits the imaging condition information 314 set in the selected imaging protocol 310 to the radiation detection device 120-2 and the synchronization control device 140. Furthermore, upon receiving the imaging condition information 314 from the integrated control device 130, the synchronization control device 140 notifies the radiation generation device 110-2 of the required imaging condition information.

In step S22, the integrated control device 130 periodically acquires the information indicating the orientation of the radiation generation device 110-2 from the orientation detection sensor 111 of the radiation generation device 110-2. According to the second embodiment, the determination unit 132 of the integrated control device 130 is a determination unit for determining whether radiography of the subject 12 can be performed using the information indicating the orientation of the radiation generation device 110-2 acquired from the orientation detection sensor 111. More specifically, the determination unit 132 compares the orientation indicated by the orientation information 313 in the imaging protocol 310 selected in step S21 with the orientation of the radiation generation device 110-2 acquired from the orientation detection sensor 111 and determines whether radiography can be performed on the basis of the result of the comparison. Still more specifically, the determination unit 132 determines that radiography can be performed if the difference between the orientation indicated by the orientation information 313 in the imaging protocol 310 selected in step S21 and the orientation of the radiation generation device 110-2 acquired from the orientation detection sensor 111 is within a predetermined range. However, the determination unit 132 determines that radiography cannot be performed if the difference between the orientation indicated by the orientation information 313 in the imaging protocol 310 selected in step S21 and the orientation of the radiation generation device 110-2 acquired from the orientation detection sensor 111 is not within the predetermined range.

If the determination unit 132 determines that radiography of the subject 12 cannot be performed, the integrated control device 130 notifies the synchronization control device 140 that radiography cannot be performed. When the determination unit 132 determines that radiography of the subject 12 cannot be performed, the integrated control device 130 causes the display device 180 to display “Radiography not available” as information indicating the result of determination as to whether radiography can be performed. Furthermore, the integrated control device 130 causes the display device 180 to display the information indicating the difference between the orientation of the radiation generation device 110-2 in the imaging protocol 310 and the orientation detected by the orientation detection sensor 111.

However, if the determination unit 132 determines that radiography of the subject 12 can be performed, the integrated control device 130 notifies the synchronization control device 140 that radiography can be performed. In addition, when the determination unit 132 determines that radiography of the subject 12 can be performed, the integrated control device 130 causes the display device 180 to display “Radiography available” as information indicating the result of determination as to whether radiography can be performed. Still furthermore, the integrated control device 130 causes the display device 180 to display the information indicating the difference between the orientation of the radiation generation device 110-2 in the imaging protocol 310 and the orientation detected by the orientation detection sensor 111.

As illustrated in FIG. 8, in step S22, the determination unit 132 determines whether radiography of the subject 12 can be performed each time the information indicating the orientation of the radiation generation device 110-2 is acquired from the orientation detection sensor 111 of the radiation generation device 110-2.

In step S23, when the exposure switch 150 is pressed by the radiographer 11, the synchronization control device 140 detects this event. The synchronization control device 140 then controls emission of radiation from the radiation generation device 110-2 according to the result of determination as to whether radiography can be performed at the time the exposure switch 150 is pressed. More specifically, if the result of determination as to whether radiography can be performed at the time the exposure switch 150 is pressed is “Radiography not available,” the synchronization control device 140 performs control to continue the stoppage of emission of radiation from the radiation generation device 110-2 without sending any notification to the radiation generation device 110-2. However, if the result of determination as to whether radiography can be performed at the time the exposure switch 150 is pressed is “Radiography available,” the synchronization control device 140 notifies the radiation generation device 110-2 of the start of radiation exposure and performs control to start the emission of radiation from the radiation generation device 110-2. In addition, if the result of determination as to whether radiography can be performed at the time the exposure switch 150 is pressed is “Radiography available”, the synchronization control device 140 notifies the radiation detection device 120-2 of the start of retrieving a radiation image via the integrated control device 130. Thereafter, upon completion of retrieving the radiation image, the radiation detection device 120-2 transmits the retrieved radiation image to the integrated control device 130.

When the processing in step S23 is completed, the process of the sequence diagram illustrated in FIG. 8 ends.

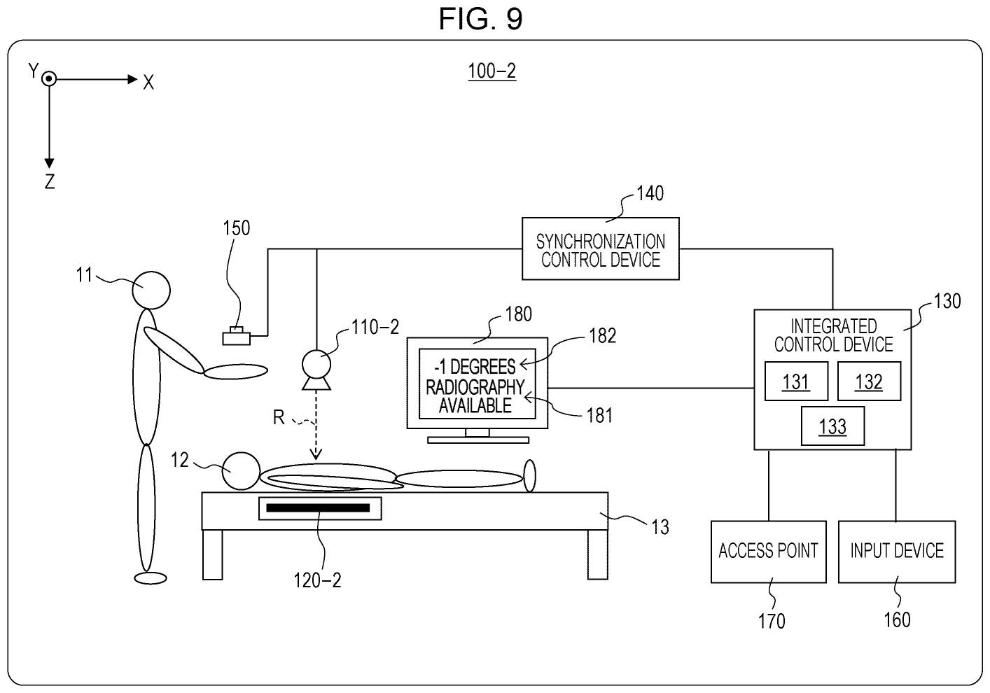

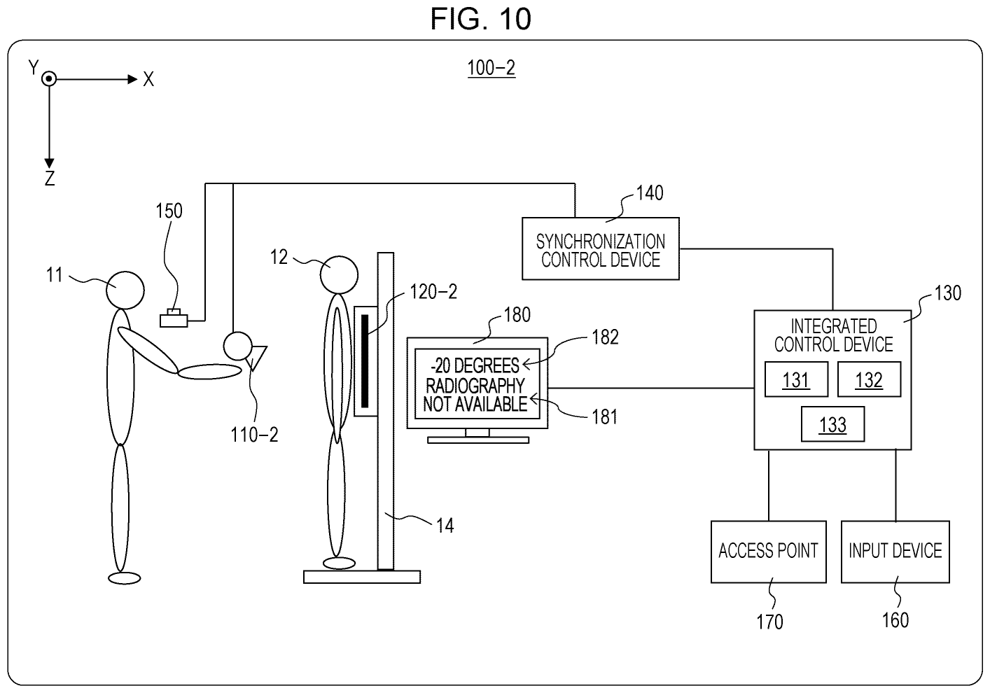

The processing performed for each of the plurality of imaging protocols 310 illustrated in FIG. 7 is described below with reference to FIGS. 9 and 10.

FIG. 9 illustrates an example of the radiography performed by the radiography system 100-2 using the imaging protocol 310 having the imaging protocol number information 311 being “21” illustrated in FIG. 7, according to the second embodiment of the present disclosure. In FIG. 9, the configurations similar to those illustrated in FIGS. 1 and 8 are identified by the same reference numerals, and detailed descriptions of the configurations are omitted. FIG. 9 also illustrates the XYZ coordinate system corresponding to the XYZ coordinate system illustrated in FIG. 1. In FIG. 9, as illustrated in FIG. 8, the radiation generation device 110-2 includes the orientation detection sensor 111 for detecting the orientation of the radiation generation device 110-2. Furthermore, in FIG. 9, the radiographer 11 installs the radiation detection device 120-2 and adjusts the orientation of the radiation generation device 110-2.

In the imaging protocol 310 having the imaging protocol number information 311 being “21” illustrated in FIG. 7, “lying position” is set as the radiographic positioning information 312, “0 degrees” is set as the orientation information 313 of the radiation generation device 110-2, and “imaging conditions 21” is set as the imaging condition information 314. The imaging protocol 310 having the imaging protocol number information 311 being “21” illustrated in FIG. 7 is used when the radiation detection device 120-2 is fixed to the radiographic table 13 during radiography of the subject 12 in the lying position. In the imaging protocol 310 having the imaging protocol number information 311 being “21” illustrated in FIG. 7, the orientation of the radiation generation device 110-2 is set to “0 degrees” (the Z direction) to match the orientation of the radiation detection device 120-2 fixed in the radiographic table 13. In the example illustrated in FIG. 9, the orientation of the radiation generation device 110-2 is information indicating the angle of inclination of the radiation generation device 110-2 with respect to the Z direction that indicates the vertical direction and that indicates an angle of inclination of 0 degrees (the angle measured in a counterclockwise direction from the Z direction (to the X direction) is a positive (+) angle). In the example illustrated in FIG. 9, the predetermined range used by the determination unit 132 of the integrated control device 130 to determine whether radiography can be performed is set to ±5 degrees, for example. That is, in this case, the determination unit 132 determines that radiography can be performed if the angle indicating the orientation of the radiation generation device 110-2 acquired from the orientation detection sensor 111 is in the range of-5 degrees to 5 degrees (inclusive) and determines that radiography cannot be performed if the angle is not in the range of-5 degrees to 5 degrees.

In step S21 illustrated in FIG. 8, the imaging protocol 310 having the imaging protocol number information 311 being “21” illustrated in FIG. 7 is selected, and radiography is started. In this case, in step S22 illustrated in FIG. 8, the determination unit 132 of the integrated control device 130 determines whether radiography of the subject 12 can be performed. More specifically, the determination unit 132 determines whether radiography can be performed on the basis of the difference between the orientation information 313 (0 degrees) in the imaging protocol 310 having the imaging protocol number information 311 being “21” and the orientation of the radiation generation device 110-2 acquired from the orientation detection sensor 111. In the example illustrated in FIG. 9, the orientation of the radiation generation device 110-2 acquired from the orientation detection sensor 111 is-1 degree, and it is determined that radiography can be performed on the basis of the difference between-1 degree and the orientation information 313 (0 degrees) of the imaging protocol 310 having the imaging protocol number information 311 being “21”. That is, the determination unit 132 determines that radiography of the subject 12 can be performed because the difference is −1 degree, which is in the predetermined range of ±5 degrees.

When the determination unit 132 determines that radiography of the subject 12 can be performed, the integrated control device 130 notifies the synchronization control device 140 that radiography can be performed. In addition, when the determination unit 132 determines that radiography of the subject 12 can be performed, the integrated control device 130 causes the display device 180 to display “Radiography available” as the information 181 indicating the result of determination as to whether radiography can be performed, as illustrated in FIG. 9. Furthermore, the integrated control device 130 causes the display device 180 to display “−1 degree” as the information 182 indicating the difference between the orientation of the radiation generation device 110-2 in the imaging protocol 310 and the orientation detected by the orientation detection sensor 111, as illustrated in FIG. 9. This enables the radiographer 11 to perform radiography using the exposure switch 150 after checking the information 181 (“Radiography available”) that is displayed on the display device 180 to indicate the determination result of the availability of radiography.

In step S23 illustrated in FIG. 8, when the exposure switch 150 is pressed by the radiographer 11, the synchronization control device 140 notifies the radiation generation device 110-2 of the start of radiation exposure and performs control to start emission of radiation R from the radiation generation device 110-2. In addition, since the result of determination as to whether radiography can be performed at the time the exposure switch 150 is pressed is “Radiography available,” the synchronization control device 140 notifies the radiation detection device 120-2 of the start of retrieving a radiation image via the integrated control device 130. Thereafter, upon completion of retrieving the radiation image, the radiation detection device 120-2 transmits the retrieved radiation image to the integrated control device 130.

FIG. 10 illustrates an example of the radiography performed by the radiography system 100-2 using the imaging protocol 310 having the imaging protocol number information 311 being “22” illustrated in FIG. 7, according to the second embodiment of the present disclosure. In FIG. 10, the configurations similar to those illustrated in FIGS. 1, 8, and 9 are identified by the same reference numerals, and detailed descriptions of the configurations are omitted. In the radiography system 100-2 illustrated in FIG. 10, a vertical radiographic stand 14 is installed instead of installing the radiographic table 13 of the radiography system 100-2 illustrated in FIG. 9. FIG. 10 also illustrates the XYZ coordinate system corresponding to the XYZ coordinate system illustrated in FIGS. 1 and 9. In FIG. 10, as illustrated in FIG. 8, the radiation generation device 110-2 includes the orientation detection sensor 111 for detecting the orientation of the radiation generation device 110-2. Furthermore, in FIG. 10, the radiographer 11 installs the radiation detection device 120-2 and adjusts the orientation of the radiation generation device 110-2.

In the imaging protocol 310 having the imaging protocol number information 311 being “22” illustrated in FIG. 7, “lying position” is set as the radiographic positioning information 312, “90 degrees” is set as the orientation information 313 of the radiation generation device 110-2, and “imaging conditions 22” is set as the imaging condition information 314. The imaging protocol 310 having the imaging protocol number information 311 being “22” illustrated in FIG. 7 is used when the radiation detection device 120-2 is fixed to the vertical radiographic stand 14 during radiography of the subject 12 in a standing position. In the imaging protocol 310 having the imaging protocol number information 311 being “22” illustrated in FIG. 7, the orientation of the radiation generation device 110-2 is set to 90 degrees (the X direction) so as to match the orientations of the radiation detection device 120-2 fixed to the vertical radiographic stand 14. In the example illustrated in FIG. 10, the orientation of the radiation generation device 110-2 is information indicating the angle of inclination of the radiation generation device 110-2 with respect to the Z direction that indicates the vertical direction and that indicates an angle of inclination of 0 degrees (the angle measured in a counterclockwise direction from the Z direction (to the X direction) is a positive (+) angle). In addition, in the example illustrated in FIG. 10, the predetermined range used for the determination unit 132 of the integrated control device 130 to determine whether radiography can be performed is set to ±5 degrees, for example. That is, in this case, the determination unit 132 determines that radiography can be performed if the angle indicating the orientation of the radiation generation device 110-2 acquired from the orientation detection sensor 111 is in the range of 85 degrees to 95 degrees (inclusive) and determines that radiography cannot be performed if the angle is not in the range of 85 degrees to 95 degrees.

In step S21 illustrated in FIG. 8, the imaging protocol 310 having the imaging protocol number information 311 being “22” illustrated in FIG. 7 is selected, and radiography is started. In this case, in step S22 illustrated in FIG. 8, the determination unit 132 of the integrated control device 130 determines whether radiography of the subject 12 can be performed. More specifically, the determination unit 132 determines whether radiography can be performed on the basis of the difference between the orientation information 313 (90 degrees) in the imaging protocol 310 having the imaging protocol number information 311 being “22” and the orientation of the radiation generation device 110-2 acquired from the orientation detection sensor 111. In the example illustrated in FIG. 10, the orientation of the radiation generation device 110-2 acquired from the orientation detection sensor 111 is 70 degrees, and it is determined that radiography cannot be performed on the basis of the difference between 70 degrees and the orientation information 313 (90 degrees) of the imaging protocol 310 having the imaging protocol number information 311 being “22”. That is, the determination unit 132 determines that radiography of the subject 12 cannot be performed because the difference is-20 degrees, which is not in the predetermined range of ±5 degrees.

When the determination unit 132 determines that radiography of the subject 12 cannot be performed, the integrated control device 130 notifies the synchronization control device 140 that radiography cannot be performed. In addition, when the determination unit 132 determines that radiography of the subject 12 cannot be performed, the integrated control device 130 causes the display device 180 to display “Radiography not available” as the information 181 indicating the result of determination as to whether radiography can be performed, as illustrated in FIG. 10. Furthermore, the integrated control device 130 causes the display device 180 to display “−20 degrees” as the information 182 indicating the difference between the orientation of the radiation generation device 110-2 in the imaging protocol 310 and the orientation detected by the orientation detection sensor 111, as illustrated in FIG. 10. This enables the radiographer 11 to adjust the installation position of the radiation generation device 110-2 after checking the information 181 (“Radiography not available”) that is displayed on the display device 180 to indicate the determination result of the availability of radiography and the information 182 indicating the difference in the orientations of the radiation generation device 110-2. That is, according to the second embodiment, the positioning of the radiation generation device 110-2 necessary for radiography can be performed with a simple configuration.

Thereafter, when the radiographer 11 adjusts the installation position of the radiation generation device 110-2 so that the angle indicating the orientation of the radiation generation device 110-2 acquired from the orientation detection sensor 111 is in the range of 85 degrees to 95 degrees, the difference described above is included in the predetermined range (±5 degrees). In this case, the determination unit 132 determines that radiography of the subject 12 can be performed. When the determination unit 132 determines that radiography of the subject 12 can be performed, the integrated control device 130 notifies the synchronization control device 140 that radiography can be performed. In addition, when the determination unit 132 determines that radiography of the subject 12 can be performed, the integrated control device 130 causes the display device 180 to display “Radiography available” as the information 181 indicating the result of determination as to whether radiography can be performed. Furthermore, the integrated control device 130 causes the display device 180 to display the information 182 indicating the difference between the orientation of the radiation generation device 110-2 in the imaging protocol 310 and the orientation detected by the orientation detection sensor 111. This enables the radiographer 11 to perform radiography using the exposure switch 150 after checking the information 181 (“Radiography available”) that is displayed on the display device 180 to indicate the determination result of the availability of radiography.

In step S23 illustrated in FIG. 8, when the exposure switch 150 is pressed by the radiographer 11, the synchronization control device 140 notifies the radiation generation device 110-2 of the start of radiation exposure and performs control to start emission of radiation R from the radiation generation device 110-2. In addition, since the result of determination as to whether radiography can be performed at the time the exposure switch 150 is pressed is “Radiography available,” the synchronization control device 140 notifies the radiation detection device 120-2 of the start of retrieving a radiation image via the integrated control device 130. Thereafter, upon completion of retrieving the radiation image, the radiation detection device 120-2 transmits the retrieved radiation image to the integrated control device 130.

According to the second embodiment, the radiography system 100-2 includes the setting unit 131 that sets up the imaging protocols 310 containing the orientation of the radiation generation device 110-2 which is paired with the radiation detection device 120-2 required for radiography. The radiography system 100-2 further includes the orientation detection sensor 111, which is a detection unit for detecting the orientation of the radiation generation device 110-2. Still furthermore, the radiography system 100-2 includes the determination unit 132 that determines whether radiography can be performed on the basis of the orientation of the radiation generation device included in the imaging protocol 310 set by the setting unit 131 and the orientation of the radiation generation device 110-2 detected by the orientation detection sensor 111.

Such a simple configuration enables determination as to whether radiography can be performed.

According to the present disclosure, it is possible to determine whether radiography can be performed with a simple configuration.

### Other Embodiments

The present disclosure can also be implemented by supplying a program that provides one or more functions according to the above embodiments to a system or an apparatus via a network or a storage medium and executing the program by one or more processors of the system or the apparatus that read the program. Alternatively, the present disclosure can be implemented by a circuit (for example, ASIC) that provides the one or more functions.

The above-described program and a computer-readable storage medium storing the program are included in the present disclosure.

The above-described embodiments of the present disclosure are merely examples of embodiments for implementing the present disclosure and should not be interpreted as limiting the technical scope of the disclosure. That is, the present disclosure can be implemented in various forms without departing from the technical concept or main features thereof.

The present disclosure is not limited to the above embodiments, and various changes and modifications can be made without departing from the spirit and scope of the present disclosure. Therefore, the following claims are attached to publicly disclose the scope of the present disclosure.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

1. A radiography system for performing radiography using radiation, comprising:
a setting unit configured to set up an imaging protocol including orientation of one of a radiation generation device that generates the radiation and a radiation detection device that detects the radiation in a form of an image signal;
a detection unit configured to detect the orientation of the one device; and
a determination unit configured to determine whether the radiography can be performed based on the orientation of the one device included in the imaging protocol set up by the setting unit and the orientation of the one device detected by the detection unit.

2. The radiography system according to claim 1, wherein the determination unit determines that the radiography can be performed if a difference between the orientation of the one device included in the imaging protocol set up by the setting unit and the orientation of the one device detected by the detection unit is within a predetermined range.

3. The radiography system according to claim 1, wherein the determination unit determines that the radiography cannot be performed if a difference between the orientation of the one device included in the imaging protocol set up by the setting unit and the orientation of the one device detected by the detection unit is not within a predetermined range.

4. The radiography system according to claim 1, further comprising:
a display device configured to display information indicating a result of determination made by the determination unit as to whether the radiography can be performed.

5. The radiography system according to claim 1, further comprising:
a display device configured to display information indicating a difference between the orientation of the one device included in the imaging protocol set up by the setting unit and the orientation of the one device detected by the detection unit.

6. The radiography system according to claim 1, further comprising:
a display device configured to display the information indicating a result of determination made by the determination unit as to whether the radiography can be performed and the information indicating a difference between the orientation of the one device included in the imaging protocol set up by the setting unit and the orientation of the one device detected by the detection unit.

7. The radiography system according to claim 1, wherein the one device is the radiation detection device,
wherein the setting unit sets up the imaging protocol including the orientation of the radiation detection device, and
wherein the detection unit detects the orientation of the radiation detection device, and the determination unit determines whether the radiography can be performed based on the orientation of the radiation detection device included in the imaging protocol set up by the setting unit and the orientation of the radiation detection device detected by the detection unit.

8. The radiography system according to claim 1, wherein the one device is the radiation generation device,
wherein the setting unit sets up the imaging protocol including the orientation of the radiation generation device, and
wherein the detection unit detects the orientation of the radiation generation device, and the determination unit determines whether the radiography can be performed based on the orientation of the radiation generation device included in the imaging protocol set up by the setting unit and the orientation of the radiation generation device detected by the detection unit.

9. A radiography method for use by a radiography system, the radiography system performing radiography using radiation, the method comprising:
setting up an imaging protocol including orientation of one of a radiation generation device that generates the radiation and a radiation detection device that detects the radiation in a form of an image signal; and
detecting the orientation of the one device; and
determining whether the radiography can be performed based on the orientation of the one device included in the set-up imaging protocol and the detected orientation of the one device.

10. A non-transitory computer-readable storage medium storing one or more programs including executable instructions, which when executed by a computer, cause the computer to perform the method according to claim 9.
